SEQUENCE LISTING (12 sequences)

SEQ ID NO: 1 - protein, 12 aa, Artificial
  Synthetic Peptide
CAECWIDGWVPC

SEQ ID NO: 2 - protein, 12 aa, Artificial
  Synthetic Peptide
CGECWADGWVPC

SEQ ID NO: 3 - protein, 12 aa, Artificial
  Synthetic Peptide
CTECWVDGWVPC

SEQ ID NO: 4 - protein, 12 aa, Artificial
  Synthetic Peptide
CSECWVDGWVSC

SEQ ID NO: 5 - protein, 12 aa, Artificial
  Synthetic Peptide
CLECWVDGWVPC

SEQ ID NO: 6 - protein, 12 aa, Artificial
  Synthetic Peptide
CEECWVDGWVPC

SEQ ID NO: 7 - protein, 12 aa, Artificial
  Synthetic Peptide
CAECWIDGWVEC

SEQ ID NO: 8 - protein, 12 aa, Artificial
  Synthetic Peptide
CTECWIDGWVPC

SEQ ID NO: 9 - protein, 12 aa, Artificial
  Synthetic Peptide
CRECWVDGWVEC

SEQ ID NO: 10 - protein, 12 aa, Artificial
  Synthetic Peptide
CGECWVDGWVSC

SEQ ID NO: 11 - protein, 12 aa, Artificial
  Synthetic Peptide
CWWCEDITGRPC

SEQ ID NO: 12 - protein, 12 aa, Artificial
  Synthetic Peptide
  Xaa (2)..(3): Xaa represents any amino acid residue
  Xaa (6)..(6): Xaa is I, A or V
  Xaa (10)..(10): Xaa is V, I or M
  Xaa (11)..(11): Xaa is P, S or E
CXXCWXDGWXXC

(19) United States
(12) Patent Application Publication
(10) Pub. No.: US 2022/0133732 A1
(43) Pub. Date: May 5, 2022

(54) BICYCLIC PEPTIDE LIGANDS SPECIFIC FOR CAIX

(71) Applicant: BicycleRD Limited, Cambridge (GB)

(72) Inventors: Leonardo BALDASSARRE, Cambridge (GB); Liuhong CHEN, Cambridge (GB); Rachid LANI, Cambridge (GB); Catherine STACE, Cambridge (GB); Daniel TEUFEL, Cambridge (GB); Edward WALKER, Cambridge (GB)

(21) Appl. No.: 17/422,933

(22) PCT Filed: Jan. 15, 2020
(86) PCT No.: PCT/GB2020/050070

§ 371 Date: Jul. 14, 2021

(30) Foreign Application Priority Data
Jan. 15, 2019 (GB) 1900526.3

(51) Int. Cl. A61K 31/537; A61K 47/64

(52) U.S. Cl. CPC A61K 31/537; A61K 47/64

(57) ABSTRACT

The present invention relates to polypeptides which are covalently bound to aromatic molecular scaffolds such that two or more peptide loops are subtended between attachment points to the scaffold. In particular, the invention describes peptides which are high affinity binders of carbonic anhydrase IX (CAIX).The invention also includes dmg conjugates comprising said peptides,conjugated to one or more effector and/or functional groups, to pharmaceutical compositions comprising said peptide ligands and drug conjugates and to the use of said peptide ligands and drug conjugates in preventing, suppressing or treating a disease or disorder mediated by CAIX.

### FIELD OF THE INVENTION

The present invention relates to polypeptides which are covalently bound to aromatic molecular scaffolds such that two or more peptide loops are subtended between attachment points to the scaffold. In particular, the invention describes peptides which are high affinity binders of carbonic anhydrase IX (CAIX). The invention also includes drug conjugates comprising said peptides, conjugated to one or more effector and/or functional groups, to pharmaceutical compositions comprising said peptide ligands and drug conjugates and to the use of said peptide ligands and drug conjugates in preventing, suppressing or treating a disease or disorder mediated by CAIX.

### BACKGROUND OF THE INVENTION

Cyclic peptides are able to bind with high affinity and target specificity to protein targets and hence are an attractive molecule class for the development of therapeutics. In fact, several cyclic peptides are already successfully used in the clinic, as for example the antibacterial peptide vancomycin, the immunosuppressant drug cyclosporine or the anti-cancer drug octreotide (Driggers et al. (2008), Nat Rev Drug Discov 7 (7), 608-24). Good binding properties result from a relatively large interaction surface formed between the peptide and the target as well as the reduced conformational flexibility of the cyclic structures. Typically, macrocycles bind to surfaces of several hundred square angstrom, as for example the cyclic peptide CXCR4 antagonist CVX15 (400 Å²; Wu et al. (2007), Science 330, 1066-71), a cyclic peptide with the Arg-Gly-Asp motif binding to integrin aVb3 (355 Å²) (Xiong et al. (2002), Science 296 (5565), 151-5) or the cyclic peptide inhibitor upain-1 binding to urokinase-type plasminogen activator (603 Å²; Zhao et al. (2007), J Struct Biol 160 (1), 1-10).

Due to their cyclic configuration, peptide macrocycles are less flexible than linear peptides, leading to a smaller loss of entropy upon binding to targets and resulting in a higher binding affinity. The reduced flexibility also leads to locking target-specific conformations, increasing binding specificity compared to linear peptides. This effect has been exemplified by a potent and selective inhibitor of matrix metalloproteinase 8 (MMP-8) which lost its selectivity over other MMPs when its ring was opened (Cherney et al. (1998), J Med Chem 41 (11), 1749-51). The favorable binding properties achieved through macrocyclization are even more pronounced in multicyclic peptides having more than one peptide ring as for example in vancomycin, nisin and actinomycin.

Different research teams have previously tethered polypeptides with cysteine residues to a synthetic molecular structure (Kemp and McNamara (1985), J. Org. Chem; Timmerman et al. (2005), ChemBioChem). Meloen and co-workers had used tris(bromomethyl)benzene and related molecules for rapid and quantitative cyclisation of multiple peptide loops onto synthetic scaffolds for structural mimicry of protein surfaces (Timmerman et al. (2005), ChemBioChem). Methods for the generation of candidate drug compounds wherein said compounds are generated by linking cysteine containing polypeptides to a molecular scaffold as for example tris(bromomethyl)benzene are disclosed in WO 2004/077062 and WO 2006/078161.

Phage display-based combinatorial approaches have been developed to generate and screen large libraries of bicyclic peptides to targets of interest (Heinis et al. (2009), Nat Chem Biol 5 (7), 502-7 and WO 2009/098450). Briefly, combinatorial libraries of linear peptides containing three cysteine residues and two regions of six random amino acids (Cys-(Xaa)₆-Cys-(Xaa)₆-Cys) were displayed on phage and cyclised by covalently linking the cysteine side chains to a small molecule (tris-(bromomethyl)benzene).

### SUMMARY OF THE INVENTION

According to a first aspect of the invention, there is provided a peptide ligand specific for CAIX comprising a polypeptide comprising at least three cysteine residues, separated by at least two loop sequences, and an aromatic molecular scaffold which forms covalent bonds with the cysteine residues of the polypeptide such that at least two polypeptide loops are formed on the molecular scaffold.

According to a further aspect of the invention, there is provided a drug conjugate comprising a peptide ligand as defined herein conjugated to one or more effector and/or functional groups.

According to a further aspect of the invention, there is provided a pharmaceutical composition comprising a peptide ligand or a drug conjugate as defined herein in combination with one or more pharmaceutically acceptable excipients.

According to a further aspect of the invention, there is provided a peptide ligand or drug conjugate as defined herein for use in preventing, suppressing or treating a disease or disorder mediated by CAIX.

### DETAILED DESCRIPTION OF THE INVENTION

In one embodiment, said loop sequences comprise 2 or 7 amino acids.

In a further embodiment, said loop sequences comprise three cysteine residues separated by two loop sequences one of which consists of 2 amino acids and the other of which consists of 7 amino acids.

In one embodiment, the peptide ligand comprises an amino acid sequence selected from:

(SEQ ID NO: 12)
Cᵢ-X₁-X₂-Cᵢᵢ-W-I/A/V-D-G-W-V/I/M-P/S/E-Cᵢᵢᵢ;
or
(SEQ ID NO: 11)
Cᵢ-W-W-Cᵢᵢ-E-D-I-T-G-R-P-Cᵢᵢᵢ

wherein X₁ and X₂ represent any amino acid residue, and Cᵢ, Cᵢᵢ and Cᵢᵢᵢ represent first, second and third cysteine residues, respectively or a pharmaceutically acceptable salt thereof.

In a further embodiment, the peptide ligand of Cᵢ-X₁-X₂-Cᵢᵢ-W-/I/A/V-D-G-W-V/I/M-P/S/E-Cᵢᵢᵢ (SEQ ID NO: 12) comprises an amino acid sequence selected from any one of SEQ ID NOS: 1 to 10:

(SEQ ID NO: 1)
CᵢAECᵢᵢHWIDGWVPCᵢᵢᵢ;
(SEQ ID NO: 2)
CᵢGECᵢᵢWADGWVPCᵢᵢᵢ;
(SEQ ID NO: 3)
CᵢTECᵢᵢWVDGWVPCᵢᵢᵢ;
(SEQ ID NO: 4)
CᵢSECᵢᵢWVDGWVSCᵢᵢᵢ;
(SEQ ID NO: 5)
CᵢLECᵢᵢWVDGWVPCᵢᵢᵢ;
(SEQ ID NO: 6)
CᵢEECᵢᵢWVDGWVPCᵢᵢᵢ;
(SEQ ID NO: 7)
CᵢAECᵢᵢHWIDGWVECᵢᵢᵢ;
(SEQ ID NO: 8)
CᵢTECᵢᵢWIDGWVPCᵢᵢᵢ;
(SEQ ID NO: 9)
CᵢRECᵢᵢWVDGWVECᵢᵢᵢ;
and
(SEQ ID NO: 10)
CᵢGECᵢᵢWVDGWVSCᵢᵢᵢ;

wherein Cᵢ, Cᵢᵢ and Cᵢᵢᵢ represent first, second and third cysteine residues, respectively, or a pharmaceutically acceptable salt thereof.

In a further embodiment, the peptide ligand of Cᵢ-X₁-X₂-Cᵢᵢ-W-I/A/V-D-G-W-V/I/M-P/S/E-Cᵢᵢᵢ (SEQ ID NO: 12) comprises an amino acid sequence selected from:
A-(SEQ ID NO: 1)-A (herein referred to as 61-01-N001);
G-Sar₁₀-A-(SEQ ID NO: 1) (herein referred to as 61-01-N005);
A-(SEQ ID NO: 1)-A-Sar₆-K-Biotin (herein referred to as 61-01-N003);
A-(SEQ ID NO: 2)-A (herein referred to as 61-01-01-N001);
A-(SEQ ID NO: 3)-A (herein referred to as 61-01-02-N001);
G-Sar₁₀-A-(SEQ ID NO: 3)-A (herein referred to as 61-01-02-N002);
β-Ala-Sar₁₀-A-(SEQ ID NO: 3) (herein referred to as 61-01-02-N003);
Ac-A-(SEQ ID NO: 3)-A-Sar₁₀-D-K (herein referred to as 61-01-02-N004);
DOTA-β-Ala-Sar₁₀-A-(SEQ ID NO: 3) (herein referred to as 61-01-02-N006);
A-(SEQ ID NO: 4)-A (herein referred to as 61-01-03-N001);
A-(SEQ ID NO: 5)-A (herein referred to as 61-01-04-N001);
G-Sar₁₀-A-(SEQ ID NO: 5) (herein referred to as 61-01-04-N002);
A-(SEQ ID NO: 6)-A (herein referred to as 61-01-05-N001);
A-(SEQ ID NO: 7)-A (herein referred to as 61-01-06-N001);
A-(SEQ ID NO: 8)-A (herein referred to as 61-01-07-N001);
A-(SEQ ID NO: 9)-A (herein referred to as 61-01-08-N001); and
A-(SEQ ID NO: 10)-A (herein referred to as 61-01-09-N001).

In one embodiment, the molecular scaffold is selected from 1,3,5-tris(bromomethyl)benzene (TBMB) and the peptide ligand comprises an amino acid sequence selected from:
A-(SEQ ID NO: 1)-A (herein referred to as 61-01-N001);
G-Sar₁₀-A-(SEQ ID NO: 1) (herein referred to as 61-01-N005);
A-(SEQ ID NO: 1)-A-Sar₆-K-Biotin (herein referred to as 61-01-N003);
A-(SEQ ID NO: 3)-A (herein referred to as 61-01-02-N001);
G-Sar₁₀-A-(SEQ ID NO: 3)-A (herein referred to as 61-01-02-N002);
β-Ala-Sar₁₀-A-(SEQ ID NO: 3) (herein referred to as 61-01-02-N003);
Ac-A-(SEQ ID NO: 3)-A-Sar₁₀-D-K (herein referred to as 61-01-02-N004);
DOTA-β-Ala-Sar₁₀-A-(SEQ ID NO: 3) (herein referred to as 61-01-02-N006);
A-(SEQ ID NO: 4)-A (herein referred to as 61-01-03-N001);
A-(SEQ ID NO: 5)-A (herein referred to as 61-01-04-N001);
G-Sar₁₀-A-(SEQ ID NO: 5) (herein referred to as 61-01-04-N002);
A-(SEQ ID NO: 6)-A (herein referred to as 61-01-05-N001);
A-(SEQ ID NO: 7)-A (herein referred to as 61-01-06-N001);
A-(SEQ ID NO: 8)-A (herein referred to as 61-01-07-N001);
A-(SEQ ID NO: 9)-A (herein referred to as 61-01-08-N001);
A-(SEQ ID NO: 10)-A (herein referred to as 61-01-09-N001); and
A-(SEQ ID NO: 11)-A (herein referred to as 61-18-N001).

In a further embodiment, the molecular scaffold is selected from 1,3,5-tris(bromomethyl)benzene (TBMB) and the peptide ligand comprises an amino acid sequence selected from:
A-(SEQ ID NO: 1)-A (herein referred to as 61-01-N001);
G-Sar₁₀-A-(SEQ ID NO: 1) (herein referred to as 61-01-N005);
A-(SEQ ID NO: 1)-A-Sar₆-K-Biotin (herein referred to as 61-01-N003);
A-(SEQ ID NO: 3)-A (herein referred to as 61-01-02-N001);
G-Sar₁₀-A-(SEQ ID NO: 3)-A (herein referred to as 61-01-02-N002);
β-Ala-Sar₁₀-A-(SEQ ID NO: 3) (herein referred to as 61-01-02-N003);
Ac-A-(SEQ ID NO: 3)-A-Sar₁₀-D-K (herein referred to as 61-01-02-N004);
DOTA-β-Ala-Sar₁₀-A-(SEQ ID NO: 3) (herein referred to as 61-01-02-N006);
A-(SEQ ID NO: 4)-A (herein referred to as 61-01-03-N001);
A-(SEQ ID NO: 5)-A (herein referred to as 61-01-04-N001);
G-Sar₁₀-A-(SEQ ID NO: 5) (herein referred to as 61-01-04-N002);
A-(SEQ ID NO: 6)-A (herein referred to as 61-01-05-N001);
A-(SEQ ID NO: 7)-A (herein referred to as 61-01-06-N001);
A-(SEQ ID NO: 8)-A (herein referred to as 61-01-07-N001);
A-(SEQ ID NO: 9)-A (herein referred to as 61-01-08-N001); and
A-(SEQ ID NO: 10)-A (herein referred to as 61-01-09-N001).

The scaffold/peptide ligands of this embodiment demonstrated superior CAIX competition binding as shown herein in Table 1.

In a further embodiment, the molecular scaffold is selected from 1,3,5-tris(bromomethyl)benzene (TBMB) and the peptide ligand comprises an amino acid sequence selected from:
A-(SEQ ID NO: 3)-A (herein referred to as 61-01-02-N001); and
A-(SEQ ID NO: 5)-A (herein referred to as 61-01-04-N001).

The scaffold/peptide ligands of this embodiment demonstrated superior CAIX competition binding as shown herein in Table 1 in addition to good levels of enzyme inhibition.

In a yet further embodiment, the molecular scaffold is selected from 1,3,5-tris(bromomethyl)benzene (TBMB) and the peptide ligand comprises an amino acid sequence selected from:
β-Ala-Sar₁₀-A-(SEQ ID NO: 3) (herein referred to as 61-01-02-N003).

The scaffold/peptide ligand of this embodiment demonstrated superior CAIX competition binding alone (as shown herein in Table 1) and when conjugated to the toxin DM-1 (as shown herein in Table 2).

Unless defined otherwise, all technical and scientific terms used herein have the same meaning as commonly understood by those of ordinary skill in the art, such as in the arts of peptide chemistry, cell culture and phage display, nucleic acid chemistry and biochemistry. Standard techniques are used for molecular biology, genetic and biochemical methods (see Sambrook et al., Molecular Cloning: A Laboratory Manual, 3rd ed., 2001, Cold Spring Harbor Laboratory Press, Cold Spring Harbor, N.Y.; Ausubel et al., Short Protocols in Molecular Biology (1999) 4^{th} ed., John Wiley & Sons, Inc.), which are incorporated herein by reference.

Nomenclature

Numbering

When referring to amino acid residue positions within the peptides of the invention, cysteine residues (Cᵢ, Cᵢᵢ and Cᵢᵢᵢ) are omitted from the numbering as they are invariant, therefore, the numbering of amino acid residues within the peptides of the invention is referred to as below:
-Cᵢ-A₁-E₂-Cᵢᵢ-W₃-I₄-D₅-G₆-W₇-V₈-P₉-Cᵢᵢᵢ-(SEQ ID NO: 1).

For the purpose of this description, all bicyclic peptides are assumed to be cyclised with TBMB (1,3,5-tris(bromomethyl)benzene) and yielding a tri-substituted structure. Cyclisation with TBMB occurs on Cᵢ, Cᵢᵢ, and Cᵢᵢᵢ.

Molecular Format

N- or C-terminal extensions to the bicycle core sequence are added to the left or right side of the sequence, separated by a hyphen. For example, an N-terminal βAla-Sar10-Ala tail would be denoted as:
βAla-Sar10-A-(SEQ ID NO: X).

Inversed Peptide Sequences

In light of the disclosure in Nair et al (2003) J Immunol 170(3), 1362-1373, it is envisaged that the peptide sequences disclosed herein would also find utility in their retro-inverso form. For example, the sequence is reversed (i.e. N-terminus becomes C-terminus and vice versa) and their stereochemistry is likewise also reversed (i.e. D-amino acids become L-amino acids and vice versa).

Peptide Ligands

A peptide ligand, as referred to herein, refers to a peptide covalently bound to a molecular scaffold. Typically, such peptides comprise two or more reactive groups (i.e. cysteine residues) which are capable of forming covalent bonds to the scaffold, and a sequence subtended between said reactive groups which is referred to as the loop sequence, since it forms a loop when the peptide is bound to the scaffold. In the present case, the peptides comprise at least three cysteine residues (referred to herein as Cᵢ, Cᵢᵢ and Cᵢᵢᵢ), and form at least two loops on the scaffold.

Advantages of the Peptide Ligands

Certain bicyclic peptides of the present invention have a number of advantageous properties which enable them to be considered as suitable drug-like molecules for injection, inhalation, nasal, ocular, oral or topical administration. Such advantageous properties include:
Species cross-reactivity. This is a typical requirement for preclinical pharmacodynamics and pharmacokinetic evaluation;
Protease stability. Bicyclic peptide ligands should ideally demonstrate stability to plasma proteases, epithelial (“membrane-anchored”) proteases, gastric and intestinal proteases, lung surface proteases, intracellular proteases and the like. Protease stability should be maintained between different species such that a bicycle lead candidate can be developed in animal models as well as administered with confidence to humans;
Desirable solubility profile. This is a function of the proportion of charged and hydrophilic versus hydrophobic residues and intra/inter-molecular H-bonding, which is important for formulation and absorption purposes;
An optimal plasma half-life in the circulation. Depending upon the clinical indication and treatment regimen, it may be required to develop a bicyclic peptide for short exposure in an acute illness management setting, or develop a bicyclic peptide with enhanced retention in the circulation, and is therefore optimal for the management of more chronic disease states. Other factors driving the desirable plasma half-life are requirements of sustained exposure for maximal therapeutic efficiency versus the accompanying toxicology due to sustained exposure of the agent; and
Selectivity. Certain peptide ligands of the invention demonstrate good selectivity over other carbonic anhydrases.

Pharmaceutically Acceptable Salts

It will be appreciated that salt forms are within the scope of this invention, and references to peptide ligands include the salt forms of said ligands.

The salts of the present invention can be synthesized from the parent compound that contains a basic or acidic moiety by conventional chemical methods such as methods described in Pharmaceutical Salts: Properties, Selection, and Use, P. Heinrich Stahl (Editor), Camille G. Wermuth (Editor), ISBN: 3-90639-026-8, Hardcover, 388 pages, August 2002.

Generally, such salts can be prepared by reacting the free acid or base forms of these compounds with the appropriate base or acid in water or in an organic solvent, or in a mixture of the two.

Acid addition salts (mono- or di-salts) may be formed with a wide variety of acids, both inorganic and organic. Examples of acid addition salts include mono- or di-salts formed with an acid selected from the group consisting of acetic, 2,2-dichloroacetic, adipic, alginic, ascorbic (e.g. L-ascorbic), L-aspartic, benzenesulfonic, benzoic, 4-acetamidobenzoic, butanoic, (+) camphoric, camphor-sulfonic, (+)-(1S)-camphor-10-sulfonic, capric, caproic, caprylic, cinnamic, citric, cyclamic, dodecylsulfuric, ethane-1,2-disulfonic, ethanesulfonic, 2-hydroxyethanesulfonic, formic, fumaric, galactaric, gentisic, glucoheptonic, D-gluconic, glucuronic (e.g. D-glucuronic), glutamic (e.g. L-glutamic), a-oxoglutaric, glycolic, hippuric, hydrohalic acids (e.g. hydrobromic, hydrochloric, hydriodic), isethionic, lactic (e.g. (+)-L-lactic, (±)-DL-lactic), lactobionic, maleic, malic, (−)-L-malic, malonic, (±)-DL-mandelic, methanesulfonic, naphthalene-2-sulfonic, naphthalene-1,5-disulfonic, 1-hydroxy-2-naphthoic, nicotinic, nitric, oleic, orotic, oxalic, palmitic, pamoic, phosphoric, propionic, pyruvic, L-pyroglutamic, salicylic, 4-amino-salicylic, sebacic, stearic, succinic, sulfuric, tannic, (+)-L-tartaric, thiocyanic, p-toluenesulfonic, undecylenic and valeric acids, as well as acylated amino acids and cation exchange resins.

One particular group of salts consists of salts formed from acetic, hydrochloric, hydriodic, phosphoric, nitric, sulfuric, citric, lactic, succinic, maleic, malic, isethionic, fumaric, benzenesulfonic, toluenesulfonic, sulfuric, methanesulfonic (mesylate), ethanesulfonic, naphthalenesulfonic, valeric, propanoic, butanoic, malonic, glucuronic and lactobionic acids. One particular salt is the hydrochloride salt. Another particular salt is the acetate salt.

If the compound is anionic, or has a functional group which may be anionic (e.g., —COOH may be —COO⁻), then a salt may be formed with an organic or inorganic base, generating a suitable cation. Examples of suitable inorganic cations include, but are not limited to, alkali metal ions such as Li⁺, Na⁺ and K⁺, alkaline earth metal cations such as Ca²⁺ and Mg²⁺, and other cations such as Al³⁺ or Zn⁺. Examples of suitable organic cations include, but are not limited to, ammonium ion (i.e., NH₄⁺) and substituted ammonium ions (e.g., NH₃R⁺, NH₂R₂⁺, NHR₃⁺, NR₄⁺). Examples of some suitable substituted ammonium ions are those derived from: methylamine, ethylamine, diethylamine, propylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺.

Where the peptides of the invention contain an amine function, these may form quaternary ammonium salts, for example by reaction with an alkylating agent according to methods well known to the skilled person. Such quaternary ammonium compounds are within the scope of the peptides of the invention.

Modified Derivatives

It will be appreciated that modified derivatives of the peptide ligands as defined herein are within the scope of the present invention. Examples of such suitable modified derivatives include one or more modifications selected from: N-terminal and/or C-terminal modifications; replacement of one or more amino acid residues with one or more non-natural amino acid residues (such as replacement of one or more polar amino acid residues with one or more isosteric or isoelectronic amino acids; replacement of one or more non-polar amino acid residues with other non-natural isosteric or isoelectronic amino acids); addition of a spacer group; replacement of one or more oxidation sensitive amino acid residues with one or more oxidation resistant amino acid residues; replacement of one or more amino acid residues with an alanine, replacement of one or more L-amino acid residues with one or more D-amino acid residues; N-alkylation of one or more amide bonds within the bicyclic peptide ligand; replacement of one or more peptide bonds with a surrogate bond; peptide backbone length modification; substitution of the hydrogen on the alpha-carbon of one or more amino acid residues with another chemical group, modification of amino acids such as cysteine, lysine, glutamate/aspartate and tyrosine with suitable amine, thiol, carboxylic acid and phenol-reactive reagents so as to functionalise said amino acids, and introduction or replacement of amino acids that introduce orthogonal reactivities that are suitable for functionalisation, for example azide or alkyne-group bearing amino acids that allow functionalisation with alkyne or azide-bearing moieties, respectively.

In one embodiment, the modified derivative comprises an N-terminal and/or C-terminal modification. In a further embodiment, wherein the modified derivative comprises an N-terminal modification using suitable amino-reactive chemistry, and/or C-terminal modification using suitable carboxy-reactive chemistry. In a further embodiment, said N-terminal or C-terminal modification comprises addition of an effector group, including but not limited to a cytotoxic agent, a radiochelator or a chromophore.

In a further embodiment, the modified derivative comprises an N-terminal modification. In a further embodiment, the N-terminal modification comprises an N-terminal acetyl group. In this embodiment, the N-terminal cysteine group (the group referred to herein as Cᵢ) is capped with acetic anhydride or other appropriate reagents during peptide synthesis leading to a molecule which is N-terminally acetylated. This embodiment provides the advantage of removing a potential recognition point for aminopeptidases and avoids the potential for degradation of the bicyclic peptide.

In an alternative embodiment, the N-terminal modification comprises the addition of a molecular spacer group which facilitates the conjugation of effector groups and retention of potency of the bicyclic peptide to its target.

In a further embodiment, the modified derivative comprises a C-terminal modification. In a further embodiment, the C-terminal modification comprises an amide group. In this embodiment, the C-terminal cysteine group (the group referred to herein as Cᵢᵢᵢ) is synthesized as an amide during peptide synthesis leading to a molecule which is C-terminally amidated. This embodiment provides the advantage of removing a potential recognition point for carboxypeptidase and reduces the potential for proteolytic degradation of the bicyclic peptide.

In one embodiment, the modified derivative comprises replacement of one or more amino acid residues with one or more non-natural amino acid residues. In this embodiment, non-natural amino acids may be selected having isosteric/isoelectronic side chains which are neither recognised by degradative proteases nor have any adverse effect upon target potency.

Alternatively, non-natural amino acids may be used having constrained amino acid side chains, such that proteolytic hydrolysis of the nearby peptide bond is conformationally and sterically impeded. In particular, these concern proline analogues, bulky sidechains, Cα-disubstituted derivatives (for example, aminoisobutyric acid, Aib), and cyclo amino acids, a simple derivative being amino-cyclopropylcarboxylic acid.

In one embodiment, the modified derivative comprises the addition of a spacer group. In a further embodiment, the modified derivative comprises the addition of a spacer group to the N-terminal cysteine (Cᵢ) and/or the C-terminal cysteine (Cᵢᵢᵢ).

In one embodiment, the modified derivative comprises replacement of one or more oxidation sensitive amino acid residues with one or more oxidation resistant amino acid residues. In a further embodiment, the modified derivative comprises replacement of a tryptophan residue with a naphthylalanine or alanine residue. This embodiment provides the advantage of improving the pharmaceutical stability profile of the resultant bicyclic peptide ligand.

In one embodiment, the modified derivative comprises replacement of one or more charged amino acid residues with one or more hydrophobic amino acid residues. In an alternative embodiment, the modified derivative comprises replacement of one or more hydrophobic amino acid residues with one or more charged amino acid residues. The correct balance of charged versus hydrophobic amino acid residues is an important characteristic of the bicyclic peptide ligands. For example, hydrophobic amino acid residues influence the degree of plasma protein binding and thus the concentration of the free available fraction in plasma, while charged amino acid residues (in particular arginine) may influence the interaction of the peptide with the phospholipid membranes on cell surfaces. The two in combination may influence half-life, volume of distribution and exposure of the peptide drug, and can be tailored according to the clinical endpoint. In addition, the correct combination and number of charged versus hydrophobic amino acid residues may reduce irritation at the injection site (if the peptide drug has been administered subcutaneously).

In one embodiment, the modified derivative comprises replacement of one or more L-amino acid residues with one or more D-amino acid residues. This embodiment is believed to increase proteolytic stability by steric hindrance and by a propensity of D-amino acids to stabilise β-turn conformations (Tugyi et al (2005) PNAS, 102(2), 413-418).

In one embodiment, the modified derivative comprises removal of any amino acid residues and substitution with alanines. This embodiment provides the advantage of removing potential proteolytic attack site(s).

It should be noted that each of the above mentioned modifications serve to deliberately improve the potency or stability of the peptide. Further potency improvements based on modifications may be achieved through the following mechanisms:
Incorporating hydrophobic moieties that exploit the hydrophobic effect and lead to lower off rates, such that higher affinities are achieved;
Incorporating charged groups that exploit long-range ionic interactions, leading to faster on rates and to higher affinities (see for example Schreiber et al, Rapid, electrostatically assisted association of proteins (1996), Nature Struct. Biol. 3,427-31); and
Incorporating additional constraint into the peptide, by for example constraining side chains of amino acids correctly such that loss in entropy is minimal upon target binding, constraining the torsional angles of the backbone such that loss in entropy is minimal upon target binding and introducing additional cyclisations in the molecule for identical reasons.

(for reviews see Gentilucci et al, Curr. Pharmaceutical Design, (2010), 16, 3185-203, and Nestor et al, Curr. Medicinal Chem (2009), 16, 4399-418).

Isotopic Variations

The present invention includes all pharmaceutically acceptable (radio)isotope-labeled peptide ligands of the invention, wherein one or more atoms are replaced by atoms having the same atomic number, but an atomic mass or mass number different from the atomic mass or mass number usually found in nature, and peptide ligands of the invention, wherein metal chelating groups are attached (termed “effector”) that are capable of holding relevant (radio)isotopes, and peptide ligands of the invention, wherein certain functional groups are covalently replaced with relevant (radio)isotopes or isotopically labelled functional groups.

Examples of isotopes suitable for inclusion in the peptide ligands of the invention comprise isotopes of hydrogen, such as ²H (D) and ³H (T), carbon, such as ¹¹C, ¹³C and ¹⁴C, chlorine, such as ³⁶Cl, fluorine, such as ¹⁸F, iodine, such as 123I, ¹²⁵I and ¹³¹I, nitrogen, such as ¹³N and ¹⁵N, oxygen, such as ¹⁵O, ¹⁷O and ¹⁸O, phosphorus, such as ³²P, sulfur, such as ³⁵S, copper, such as ⁶⁴Cu, gallium, such as ⁶⁷Ga or ⁶⁸Ga, yttrium, such as ⁹⁰Y and lutetium, such as ¹⁷⁷Lu, and Bismuth, such as ²¹³Bi.

Certain isotopically-labelled peptide ligands of the invention, for example, those incorporating a radioactive isotope, are useful in drug and/or substrate tissue distribution studies, and to clinically assess the presence and/or absence of the CAIX target on diseased tissues. The peptide ligands of the invention can further have valuable diagnostic properties in that they can be used for detecting or identifying the formation of a complex between a labelled compound and other molecules, peptides, proteins, enzymes or receptors. The detecting or identifying methods can use compounds that are labelled with labelling agents such as radioisotopes, enzymes, fluorescent substances, luminous substances (for example, luminol, luminol derivatives, luciferin, aequorin and luciferase), etc. The radioactive isotopes tritium, i.e. ³H (T), and carbon-14, i.e. ¹⁴C, are particularly useful for this purpose in view of their ease of incorporation and ready means of detection.

Substitution with heavier isotopes such as deuterium, i.e. ²H (D), may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, ¹O and ¹³N, can be useful in Positron Emission Topography (PET) studies for examining target occupancy.

Isotopically-labeled compounds of peptide ligands of the invention can generally be prepared by conventional techniques known to those skilled in the art or by processes analogous to those described in the accompanying Examples using an appropriate isotopically-labeled reagent in place of the non-labeled reagent previously employed.

Aromatic Molecular Scaffold

References herein to the term “aromatic molecular scaffold” refer to any molecular scaffold as defined herein which contains an aromatic carbocyclic or heterocyclic ring system.

It will be appreciated that the aromatic molecular scaffold may comprise an aromatic moiety. Examples of suitable aromatic moieties within the aromatic scaffold include biphenylene, terphenylene, naphthalene or anthracene.

It will also be appreciated that the aromatic molecular scaffold may comprise a heteroaromatic moiety. Examples of suitable heteroaromatic moieties within the aromatic scaffold include pyridine, pyrimidine, pyrrole, furan and thiophene.

It will also be appreciated that the aromatic molecular scaffold may comprise a halomethylarene moiety, such as a bis(bromomethyl)benzene, a tris(bromomethyl)benzene, a tetra(bromomethyl)benzene or derivatives thereof.

Non-limiting examples of aromatic molecular scaffolds include: bis-, tris-, or tetra(halomethyl)benzene; bis-, tris-, or tetra(halomethyl)pyridine; bis-, tris-, or tetra(halomethyl)pyridazine; bis-, tris-, or tetra(halomethyl)pyrimidine; bis-, tris-, or tetra(halomethyl)pyrazine; bis-, tris-, or tetra(halomethyl)-1,2,3-triazine; bis-, tris-, or tetra-halomethyl)-1,2,4-triazine; bis-, tris-, or tetra(halomethyl)pyrrole, -furan, -thiophene; bis-, tris-, or tetra(halomethyl)imidazole, -oxazole, -thiazol; bis-, tris-, or tetra(halomethyl)-3H-pyrazole, -isooxazole, -isothiazol; bis-, tris-, or tetra(halomethyl)biphenylene; bis-, tris-, or tetra(halomethyl)terphenylene; 1,8-bis(halomethyl)naphthalene; bis-, tris-, or tetra(halomethyl)anthracene; and bis-, tris-, or tetra(2-halomethylphenyl)methane.

More specific examples of aromatic molecular scaffolds include: 1,2-bis(halomethyl)benzene; 3,4-bis(halomethyl)pyridine; 3,4-bis(halomethyl)pyridazine; 4,5-bis(halomethyl)pyrimidine; 4,5-bis(halomethyl)pyrazine; 4,5-bis(halomethyl)-1,2,3-triazine; 5,6-bis(halomethyl)-1,2,4-triazine; 3,4-bis(halomethyl)pyrrole, -furan, -thiophene and other regioisomers; 4,5-bis(halomethyl)imidazole, -oxazole, -thiazol; 4,5-bis(halomethyl)-3H-pyrazole, -isooxazole, -isothiazol; 2,2′-bis(halomethyl)biphenylene; 2,2″-bis(halomethyl)terphenylene; 1,8-bis(halomethyl)naphthalene; 1,10-bis(halomethyl)anthracene; bis(2-halomethylphenyl)methane; 1,2,3-tris(halomethyl)benzene; 2,3,4-tris(halomethyl)pyridine; 2,3,4-tris(halomethyl)pyridazine; 3,4,5-tris(halomethyl)pyrimidine; 4,5,6-tris(halomethyl)-1,2,3-triazine; 2,3,4-tris(halomethyl)pyrrole, -furan, -thiophene; 2,4,5-bis(halomethyl)imidazole, -oxazole, -thiazol; 3,4,5-bis(halomethyl)-1H-pyrazole, -isooxazole, -isothiazol; 2,4,2′-tris(halomethyl)biphenylene; 2,3′,2″-tris(halomethyl)terphenylene; 1,3,8-tris(halomethyl)naphthalene; 1,3,10-tris(halomethyl)anthracene; bis(2-halomethylphenyl)methane; 1,2,4,5-tetra(halomethyl)benzene; 1,2,4,5-tetra(halomethyl)pyridine; 2,4,5,6-tetra(halomethyl)pyrimidine; 2,3,4,5-tetra(halomethyl)pyrrole, -furan, -thiophene; 2,2′,6,6′-tetra(halomethyl)biphenylene; 2,2″,6,6″-tetra(halomethyl) terphenylene; 2,3,5,6-tetra(halomethyl)naphthalene and 2,3,7,8-tetra(halomethyl)anthracene; and bis(2,4-bis(halomethyl)phenyl)methane.

As noted in the foregoing documents, the molecular scaffold may be a small molecule, such as a small organic molecule.

In one embodiment the molecular scaffold may be a macromolecule. In one embodiment the molecular scaffold is a macromolecule composed of amino acids, nucleotides or carbohydrates.

In one embodiment the molecular scaffold comprises reactive groups that are capable of reacting with functional group(s) of the polypeptide to form covalent bonds.

The molecular scaffold may comprise chemical groups which form the linkage with a peptide, such as amines, thiols, alcohols, ketones, aldehydes, nitriles, carboxylic acids, esters, alkenes, alkynes, azides, anhydrides, succinimides, maleimides, alkyl halides and acyl halides.

In one embodiment, the molecular scaffold may comprise or may consist of tris(bromomethyl)benzene, especially 1,3,5-tris(bromomethyl)benzene (‘TBMB’), or a derivative thereof.

In one embodiment, the molecular scaffold is 2,4,6-tris(bromomethyl)mesitylene. This molecule is similar to 1,3,5-tris(bromomethyl)benzene but contains three additional methyl groups attached to the benzene ring. This has the advantage that the additional methyl groups may form further contacts with the polypeptide and hence add additional structural constraint.

The molecular scaffold of the invention contains chemical groups that allow functional groups of the polypeptide of the encoded library of the invention to form covalent links with the molecular scaffold. Said chemical groups are selected from a wide range of functionalities including amines, thiols, alcohols, ketones, aldehydes, nitriles, carboxylic acids, esters, alkenes, alkynes, anhydrides, succinimides, maleimides, azides, alkyl halides and acyl halides.

Scaffold reactive groups that could be used on the molecular scaffold to react with thiol groups of cysteines are alkyl halides (or also named halogenoalkanes or haloalkanes).

Examples include bromomethylbenzene (the scaffold reactive group exemplified by TBMB) or iodoacetamide. Other scaffold reactive groups that are used to selectively couple compounds to cysteines in proteins are maleimides, αβ unsaturated carbonyl containing compounds and a-halomethylcarbonyl containing compounds. Examples of maleimides which may be used as molecular scaffolds in the invention include: tris-(2-maleimidoethyl)amine, tris-(2-maleimidoethyl)benzene, tris-(maleimido)benzene. An example of an α-halomethylcarbonyl containing compound is N,N′,N″-(benzene-1,3,5-triyl)tris(2-bromoacetamide). Selenocysteine is also a natural amino acid which has a similar reactivity to cysteine and can be used for the same reactions. Thus, wherever cysteine is mentioned, it is typically acceptable to substitute selenocysteine unless the context suggests otherwise.

Effector and Functional Groups

According to a further aspect of the invention, there is provided a drug conjugate comprising a peptide ligand as defined herein conjugated to one or more effector and/or functional groups.

Effector and/or functional groups can be attached, for example, to the N and/or C termini of the polypeptide, to an amino acid within the polypeptide, or to the molecular scaffold.

Appropriate effector groups include antibodies and parts or fragments thereof. For instance, an effector group can include an antibody light chain constant region (CL), an antibody CH1 heavy chain domain, an antibody CH2 heavy chain domain, an antibody CH3 heavy chain domain, or any combination thereof, in addition to the one or more constant region domains. An effector group may also comprise a hinge region of an antibody (such a region normally being found between the CH1 and CH2 domains of an IgG molecule).

In a further embodiment of this aspect of the invention, an effector group according to the present invention is an Fc region of an IgG molecule. Advantageously, a peptide ligand-effector group according to the present invention comprises or consists of a peptide ligand Fc fusion having a tβ half-life of a day or more, two days or more, 3 days or more, 4 days or more, 5 days or more, 6 days or more or 7 days or more. Most advantageously, the peptide ligand according to the present invention comprises or consists of a peptide ligand Fc fusion having a tβ half-life of a day or more.

Functional groups include, in general, binding groups, drugs, reactive groups for the attachment of other entities, functional groups which aid uptake of the macrocyclic peptides into cells, and the like.

The ability of peptides to penetrate into cells will allow peptides against intracellular targets to be effective. Targets that can be accessed by peptides with the ability to penetrate into cells include transcription factors, intracellular signalling molecules such as tyrosine kinases and molecules involved in the apoptotic pathway. Functional groups which enable the penetration of cells include peptides or chemical groups which have been added either to the peptide or the molecular scaffold. Peptides such as those derived from such as VP22, HIV-Tat, a homeobox protein of Drosophila (Antennapedia), e.g. as described in Chen and Harrison, Biochemical Society Transactions (2007) Volume 35, part 4, p821; Gupta et al. in Advanced Drug Discovery Reviews (2004) Volume 57 9637. Examples of short peptides which have been shown to be efficient at translocation through plasma membranes include the 16 amino acid penetratin peptide from Drosophila Antennapedia protein (Derossi et al (1994) J Biol. Chem. Volume 269 p10444), the 18 amino acid ‘model amphipathic peptide’ (Oehlke et al (1998) Biochim Biophys Acts Volume 1414 p127) and arginine rich regions of the HIV TAT protein. Non peptidic approaches include the use of small molecule mimics or SMOCs that can be easily attached to biomolecules (Okuyama et al (2007) Nature Methods Volume 4 p153). Other chemical strategies to add guanidinium groups to molecules also enhance cell penetration (Elson-Scwab et al (2007) J Biol Chem Volume 282 p13585). Small molecular weight molecules such as steroids may be added to the molecular scaffold to enhance uptake into cells.

One class of functional groups which may be attached to peptide ligands includes antibodies and binding fragments thereof, such as Fab, Fv or single domain fragments. In particular, antibodies which bind to proteins capable of increasing the half-life of the peptide ligand in vivo may be used.

In one embodiment, a peptide ligand-effector group according to the invention has a tβ half-life selected from the group consisting of: 12 hours or more, 24 hours or more, 2 days or more, 3 days or more, 4 days or more, 5 days or more, 6 days or more, 7 days or more, 8 days or more, 9 days or more, 10 days or more, 11 days or more, 12 days or more, 13 days or more, 14 days or more, 15 days or more or 20 days or more. Advantageously a peptide ligand-effector group or composition according to the invention will have a tβ half life in the range 12 to 60 hours. In a further embodiment, it will have a tβ half-life of a day or more. In a further embodiment still, it will be in the range 12 to 26 hours.

In one particular embodiment of the invention, the functional group is selected from a metal chelator, which is suitable for complexing metal radioisotopes of medicinal relevance.

Possible effector groups also include enzymes, for instance such as carboxypeptidase G2 for use in enzyme/prodrug therapy, where the peptide ligand replaces antibodies in ADEPT.

In one particular embodiment of the invention, the functional group is selected from a drug, such as a cytotoxic agent for cancer therapy. Suitable examples include: alkylating agents such as cisplatin and carboplatin, as well as oxaliplatin, mechlorethamine, cyclophosphamide, chlorambucil, ifosfamide; Anti-metabolites including purine analogs azathioprine and mercaptopurine or pyrimidine analogs; plant alkaloids and terpenoids including vinca alkaloids such as Vincristine, Vinblastine, Vinorelbine and Vindesine; Podophyllotoxin and its derivatives etoposide and teniposide; Taxanes, including paclitaxel, originally known as Taxol; topoisomerase inhibitors including camptothecins: irinotecan and topotecan, and type II inhibitors including amsacrine, etoposide, etoposide phosphate, and teniposide. Further agents can include antitumour antibiotics which include the immunosuppressant dactinomycin (which is used in kidney transplantations), doxorubicin, epirubicin, bleomycin, calicheamycins, and others.

In one further particular embodiment of the invention, the cytotoxic agent is selected from maytansinoids (such as DM1) or monomethyl auristatins (such as MMAE).

DM1 is a cytotoxic agent which is a thiol-containing derivative of maytansine and has the following structure:

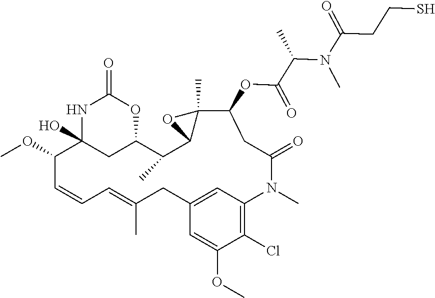 [SMILES: COc1cc2cc(c1Cl)N(C)C(=O)C[C@H](OC(=O)[C@H](C)N(C)C(=O)CCS)[C@]1(C)O[C@H]1[C@H](C)[C@@H]1C[C@@](O)(NC(=O)O1)[C@@H](OC)/C=C\C=C(/C)C2]

Monomethyl auristatin E (MMAE) is a synthetic antineoplastic agent and has the following structure:

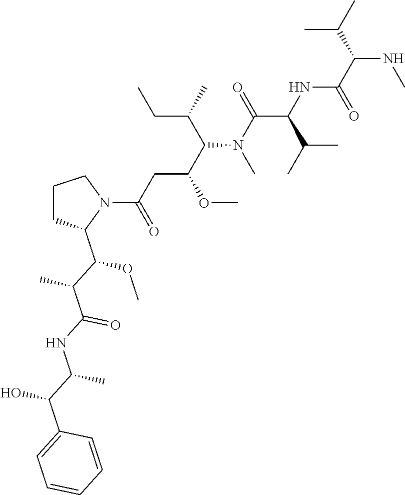

In one yet further particular embodiment of the invention, the cytotoxic agent is selected from maytansinoids (such as DM1). Data is presented herein in Table 2 which demonstrates the effects of peptide ligands conjugated to toxins containing DM1.

In one embodiment, the cytotoxic agent is linked to the bicyclic peptide by a cleavable bond, such as a disulphide bond or a protease sensitive bond. In a further embodiment, the groups adjacent to the disulphide bond are modified to control the hindrance of the disulphide bond, and by this the rate of cleavage and concomitant release of cytotoxic agent.

Published work established the potential for modifying the susceptibility of the disulphide bond to reduction by introducing steric hindrance on either side of the disulphide bond (Kellogg et al (2011) Bioconjugate Chemistry, 22, 717). A greater degree of steric hindrance reduces the rate of reduction by intracellular glutathione and also extracellular (systemic) reducing agents, consequentially reducing the ease by which toxin is released, both inside and outside the cell. Thus, selection of the optimum in disulphide stability in the circulation (which minimises undesirable side effects of the toxin) versus efficient release in the intracellular milieu (which maximises the therapeutic effect) can be achieved by careful selection of the degree of hindrance on either side of the disulphide bond.

The hindrance on either side of the disulphide bond is modulated through introducing one or more methyl groups on either the targeting entity (here, the bicyclic peptide) or toxin side of the molecular construct.

In one embodiment, the cytotoxic agent and linker is selected from any combinations of those described in WO 2016/067035 (the cytotoxic agents and linkers thereof are herein incorporated by reference).

In one embodiment, the cytotoxic agent is DM1, the bicyclic peptide is 61-01-02-N003 and the conjugate comprises a compound of formula (I):

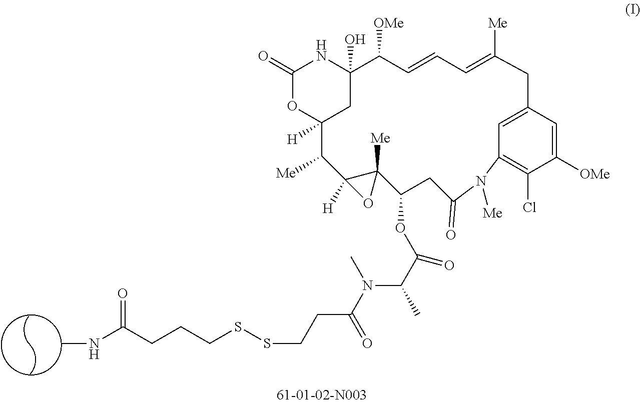 [SMILES: CNC(=O)CCCSSCCC(=O)N(C)[C@@H](C)C(=O)O[C@H]1CC(=O)N(C)c2cc(cc(OC)c2Cl)C/C(C)=C/C=C/[C@@H](OC)[C@@]2(O)C[C@H](OC(=O)N2)[C@@H](C)[C@@H]2O[C@@]12C]

The BDC of formula (I) is known herein as BT61BDC-1.

Synthesis

The peptides of the present invention may be manufactured synthetically by standard techniques followed by reaction with a molecular scaffold in vitro. When this is performed, standard chemistry may be used. This enables the rapid large scale preparation of soluble material for further downstream experiments or validation. Such methods could be accomplished using conventional chemistry such as that disclosed in Timmerman et al (supra).

Thus, the invention also relates to manufacture of polypeptides or conjugates selected as set out herein, wherein the manufacture comprises optional further steps as explained below. In one embodiment, these steps are carried out on the end product polypeptide/conjugate made by chemical synthesis.

Optionally amino acid residues in the polypeptide of interest may be substituted when manufacturing a conjugate or complex.

Peptides can also be extended, to incorporate for example another loop and therefore introduce multiple specificities.

To extend the peptide, it may simply be extended chemically at its N-terminus or C-terminus or within the loops using orthogonally protected lysines (and analogues) using standard solid phase or solution phase chemistry. Standard (bio)conjugation techniques may be used to introduce an activated or activatable N- or C-terminus. Alternatively additions may be made by fragment condensation or native chemical ligation e.g. as described in (Dawson et al. 1994. Synthesis of Proteins by Native Chemical Ligation. Science 266:776-779), or by enzymes, for example using subtiligase as described in (Chang et al Proc Natl Acad Sci U S A. 1994 Dec. 20; 91(26):12544-8 or in Hikari et al Bioorganic & Medicinal Chemistry Letters Volume 18, Issue 22, 15 Nov. 2008, Pages 6000-6003).

Alternatively, the peptides may be extended or modified by further conjugation through disulphide bonds. This has the additional advantage of allowing the first and second peptide to dissociate from each other once within the reducing environment of the cell. In this case, the molecular scaffold (e.g. TBMB) could be added during the chemical synthesis of the first peptide so as to react with the three cysteine groups; a further cysteine or thiol could then be appended to the N or C-terminus of the first peptide, so that this cysteine or thiol only reacted with a free cysteine or thiol of the second peptide, forming a disulfide—linked bicyclic peptide-peptide conjugate.

Similar techniques apply equally to the synthesis/coupling of two bicyclic and bispecific macrocycles, potentially creating a tetraspecific molecule.

Furthermore, addition of other functional groups or effector groups may be accomplished in the same manner, using appropriate chemistry, coupling at the N- or C-termini or via side chains. In one embodiment, the coupling is conducted in such a manner that it does not block the activity of either entity.

Pharmaceutical Compositions

According to a further aspect of the invention, there is provided a pharmaceutical composition comprising a peptide ligand or a drug conjugate as defined herein in combination with one or more pharmaceutically acceptable excipients.

Generally, the present peptide ligands will be utilised in purified form together with pharmacologically appropriate excipients or carriers. Typically, these excipients or carriers include aqueous or alcoholic/aqueous solutions, emulsions or suspensions, including saline and/or buffered media. Parenteral vehicles include sodium chloride solution, Ringer's dextrose, dextrose and sodium chloride and lactated Ringer's. Suitable physiologically-acceptable adjuvants, if necessary to keep a polypeptide complex in suspension, may be chosen from thickeners such as carboxymethylcellulose, polyvinylpyrrolidone, gelatin and alginates.

Intravenous vehicles include fluid and nutrient replenishers and electrolyte replenishers, such as those based on Ringer's dextrose. Preservatives and other additives, such as antimicrobials, antioxidants, chelating agents and inert gases, may also be present (Mack (1982) Remington's Pharmaceutical Sciences, 16th Edition).

The peptide ligands of the present invention may be used as separately administered compositions or in conjunction with other agents. These can include antibodies, antibody fragments and various immunotherapeutic drugs, such as cyclosporine, methotrexate, adriamycin or cisplatinum and immunotoxins. Pharmaceutical compositions can include “cocktails” of various cytotoxic or other agents in conjunction with the protein ligands of the present invention, or even combinations of selected polypeptides according to the present invention having different specificities, such as polypeptides selected using different target ligands, whether or not they are pooled prior to administration.

The route of administration of pharmaceutical compositions according to the invention may be any of those commonly known to those of ordinary skill in the art. For therapy, the peptide ligands of the invention can be administered to any patient in accordance with standard techniques. The administration can be by any appropriate mode, including parenterally, intravenously, intramuscularly, intraperitoneally, transdermally, via the pulmonary route, or also, appropriately, by direct infusion with a catheter. Preferably, the pharmaceutical compositions according to the invention will be administered by inhalation. The dosage and frequency of administration will depend on the age, sex and condition of the patient, concurrent administration of other drugs, counterindications and other parameters to be taken into account by the clinician.

The peptide ligands of this invention can be lyophilised for storage and reconstituted in a suitable carrier prior to use. This technique has been shown to be effective and art-known lyophilisation and reconstitution techniques can be employed. It will be appreciated by those skilled in the art that lyophilisation and reconstitution can lead to varying degrees of activity loss and that levels may have to be adjusted upward to compensate.

The compositions containing the present peptide ligands or a cocktail thereof can be administered for prophylactic and/or therapeutic treatments. In certain therapeutic applications, an adequate amount to accomplish at least partial inhibition, suppression, modulation, killing, or some other measurable parameter, of a population of selected cells is defined as a “therapeutically-effective dose”. Amounts needed to achieve this dosage will depend upon the severity of the disease and the general state of the patient's own immune system, but generally range from 0.005 to 5.0 mg of selected peptide ligand per kilogram of body weight, with doses of 0.05 to 2.0 mg/kg/dose being more commonly used. For prophylactic applications, compositions containing the present peptide ligands or cocktails thereof may also be administered in similar or slightly lower dosages.

A composition containing a peptide ligand according to the present invention may be utilised in prophylactic and therapeutic settings to aid in the alteration, inactivation, killing or removal of a select target cell population in a mammal. In addition, the peptide ligands described herein may be used extracorporeally or in vitro selectively to kill, deplete or otherwise effectively remove a target cell population from a heterogeneous collection of cells. Blood from a mammal may be combined extracorporeally with the selected peptide ligands whereby the undesired cells are killed or otherwise removed from the blood for return to the mammal in accordance with standard techniques.

Therapeutic Uses

The bicyclic peptides of the invention have specific utility as CAIX binding agents.

Various forms of the enzyme carbonic anhydrase (CA) catalyse hydration of carbon dioxide to generate bicarbonate anion (HCO³⁻) and a proton. Substrates of the reaction which is catalyzed by CA regulate a number of physiological processes, including formation and transport of CO₂, protons and bicarbonate anion, such as respiration, maintenance of pH levels, bone development and other processes. In the human organism, 12 catalytically active CA isoenzymes were identified which differ in their cellular localization and their expression in various tissues.

Clinical regulation of the activity of human carbonic anhydrase (hCA) by small molecular inhibitors proved to be reliable therapeutic method for a number of human diseases and already for several decades it remains a major component of therapy for high blood pressure, glaucoma, hyperthyrosis and hypoglycemia (Supuran (2008) Nat. Rev. Drug Discov. 7, 168). Classical inhibitors of carbonic anhydrases, binding into the active site of CA, are aromatic or heteroaromatic sulfonamides.

Human carbonic anhydrase IX (hCA IX) is an isoform bound to the outer cell membrane (its catalytic domain is located in the extracellular space). At physiological conditions, hCAIX is expressed only in specific tissues of gastrointestinal tract. Its overexpression was shown during hypoxia in cancer cells both in vitro and in vivo. Expression of hCAIX was detected in carcinomas of cervix, ovaries, kidneys, esophagus, lungs, breasts and brain. In tumors, hCAIX is a molecule crucial for the maintenance of intracellular pH on normal level and its expression provides the hypoxic tumor cells with an advantage in growth at acidic conditions (Chiche et al. (2009) Cancer Res 69, 358). hCAIX enzyme is thus a convenient target for development of specific inhibitors used as anti-cancer therapeutics with new mechanism of action (Neri and Supuran (2011) Nature Reviews 10, 767).

Polypeptide ligands selected according to the method of the present invention may be employed in in vivo therapeutic and prophylactic applications, in vitro and in vivo diagnostic applications, in vitro assay and reagent applications, and the like. Ligands having selected levels of specificity are useful in applications which involve testing in non-human animals, where cross-reactivity is desirable, or in diagnostic applications, where cross-reactivity with homologues or paralogues needs to be carefully controlled. In some applications, such as vaccine applications, the ability to elicit an immune response to predetermined ranges of antigens can be exploited to tailor a vaccine to specific diseases and pathogens.

Substantially pure peptide ligands of at least 90 to 95% homogeneity are preferred for administration to a mammal, and 98 to 99% or more homogeneity is most preferred for pharmaceutical uses, especially when the mammal is a human. Once purified, partially or to homogeneity as desired, the selected polypeptides may be used diagnostically or therapeutically (including extracorporeally) or in developing and performing assay procedures, immunofluorescent stainings and the like (Lefkovite and Pernis, (1979 and 1981) Immunological Methods, Volumes I and II, Academic Press, N.Y.).

According to a further aspect of the invention, there is provided a peptide ligand or a drug conjugate as defined herein, for use in preventing, suppressing or treating a disease or disorder mediated by CAIX.

According to a further aspect of the invention, there is provided a method of preventing, suppressing or treating a disease or disorder mediated by CAIX, which comprises administering to a patient in need thereof an effector group and drug conjugate of the peptide ligand as defined herein.

In one embodiment, the CAIX is mammalian CAIX. In a further embodiment, the mammalian CAIX is human CAIX (hCAIX).

In one embodiment, the disease or disorder mediated by CAIX is selected from cancer.

Examples of cancers (and their benign counterparts) which may be treated (or inhibited) include, but are not limited to tumours of epithelial origin (adenomas and carcinomas of various types including adenocarcinomas, squamous carcinomas, transitional cell carcinomas and other carcinomas) such as carcinomas of the bladder and urinary tract, breast, gastrointestinal tract (including the esophagus, stomach (gastric), small intestine, colon, rectum and anus), liver (hepatocellular carcinoma), gall bladder and biliary system, exocrine pancreas, kidney, lung (for example adenocarcinomas, small cell lung carcinomas, non-small cell lung carcinomas, bronchioalveolar carcinomas and mesotheliomas), head and neck (for example cancers of the tongue, buccal cavity, larynx, pharynx, nasopharynx, tonsil, salivary glands, nasal cavity and paranasal sinuses), ovary, fallopian tubes, peritoneum, vagina, vulva, penis, cervix, myometrium, endometrium, thyroid (for example thyroid follicular carcinoma), adrenal, prostate, skin and adnexae (for example melanoma, basal cell carcinoma, squamous cell carcinoma, keratoacanthoma, dysplastic naevus); haematological malignancies (i.e. leukemias, lymphomas) and premalignant haematological disorders and disorders of borderline malignancy including haematological malignancies and related conditions of lymphoid lineage (for example acute lymphocytic leukemia [ALL], chronic lymphocytic leukemia [CLL], B-cell lymphomas such as diffuse large B-cell lymphoma [DLBCL], follicular lymphoma, Burkitt's lymphoma, mantle cell lymphoma, T-cell lymphomas and leukaemias, natural killer [NK] cell lymphomas, Hodgkin's lymphomas, hairy cell leukaemia, monoclonal gammopathy of uncertain significance, plasmacytoma, multiple myeloma, and post-transplant lymphoproliferative disorders), and haematological malignancies and related conditions of myeloid lineage (for example acute myelogenousleukemia [AML], chronic myelogenousleukemia [CML], chronic myelomonocyticleukemia [CMML], hypereosinophilic syndrome, myeloproliferative disorders such as polycythaemia vera, essential thrombocythaemia and primary myelofibrosis, myeloproliferative syndrome, myelodysplastic syndrome, and promyelocyticleukemia); tumours of mesenchymal origin, for example sarcomas of soft tissue, bone or cartilage such as osteosarcomas, fibrosarcomas, chondrosarcomas, rhabdomyosarcomas,leiomyosarcomas, liposarcomas, angiosarcomas, Kaposi's sarcoma, Ewing's sarcoma, synovial sarcomas, epithelioid sarcomas, gastrointestinal stromal tumours, benign and malignant histiocytomas, and dermatofibrosarcomaprotuberans; tumours of the central or peripheral nervous system (for example astrocytomas, gliomas and glioblastomas, meningiomas, ependymomas, pineal tumours and schwannomas); endocrine tumours (for example pituitary tumours, adrenal tumours, islet cell tumours, parathyroid tumours, carcinoid tumours and medullary carcinoma of the thyroid); ocular and adnexal tumours (for example retinoblastoma); germ cell and trophoblastic tumours (for example teratomas, seminomas, dysgerminomas, hydatidiform moles and choriocarcinomas); and paediatric and embryonal tumours (for example medulloblastoma, neuroblastoma, Wilms tumour, and primitive neuroectodermal tumours); or syndromes, congenital or otherwise, which leave the patient susceptible to malignancy (for example Xeroderma Pigmentosum).

In a further embodiment, the cancer is selected from cancer of the cervix, ovary, kidney, esophagus, lung, breast and brain.

References herein to the term “prevention” involves administration of the protective composition prior to the induction of the disease. “Suppression” refers to administration of the composition after an inductive event, but prior to the clinical appearance of the disease. “Treatment” involves administration of the protective composition after disease symptoms become manifest.

Animal model systems which can be used to screen the effectiveness of the peptide ligands in protecting against or treating the disease are available. The use of animal model systems is facilitated by the present invention, which allows the development of polypeptide ligands which can cross react with human and animal targets, to allow the use of animal models.

The invention is further described below with reference to the following examples.

### EXAMPLES

Materials and Methods

Peptide Synthesis

Peptide synthesis was based on Fmoc chemistry, using a Symphony peptide synthesiser manufactured by Peptide Instruments and a Syro II synthesiser by MultiSynTech. Standard Fmoc-amino acids were employed (Sigma, Merck), with appropriate side chain protecting groups: where applicable standard coupling conditions were used in each case, followed by deprotection using standard methodology. Peptides were purified using HPLC and following isolation they were modified with 1,3,5-tris(bromomethyl)benzene (TBMB, Sigma). For this, linear peptide was diluted with H₂O up to -35 mL, -500 μL of 100 mM TBMB in acetonitrile was added, and the reaction was initiated with 5 mL of 1 M NH₄HCO₃ in H₂O. The reaction was allowed to proceed for ˜30 -60 min at RT, and lyophilised once the reaction had completed (judged by MALDI). Following lyophilisation, the modified peptide was purified as above, while replacing the Luna C8 with a Gemini C18 column (Phenomenex), and changing the acid to 0.1% trifluoroacetic acid. Pure fractions containing the correct TMB-modified material were pooled, lyophilised and kept at −20° C. for storage.

All amino acids, unless noted otherwise, were used in the L-configurations.

Preparation of Bicyclic Peptide Drug Conjugate BT61BDC-1

The following methodology describes the conjugation of a bicyclic peptide, namely 61-01-02-N003 (β-Ala-Sar₁₀-A-(SEQ ID NO: 3)), to a cytotoxic agent such as DM-1 to generate BT61BDC-1.

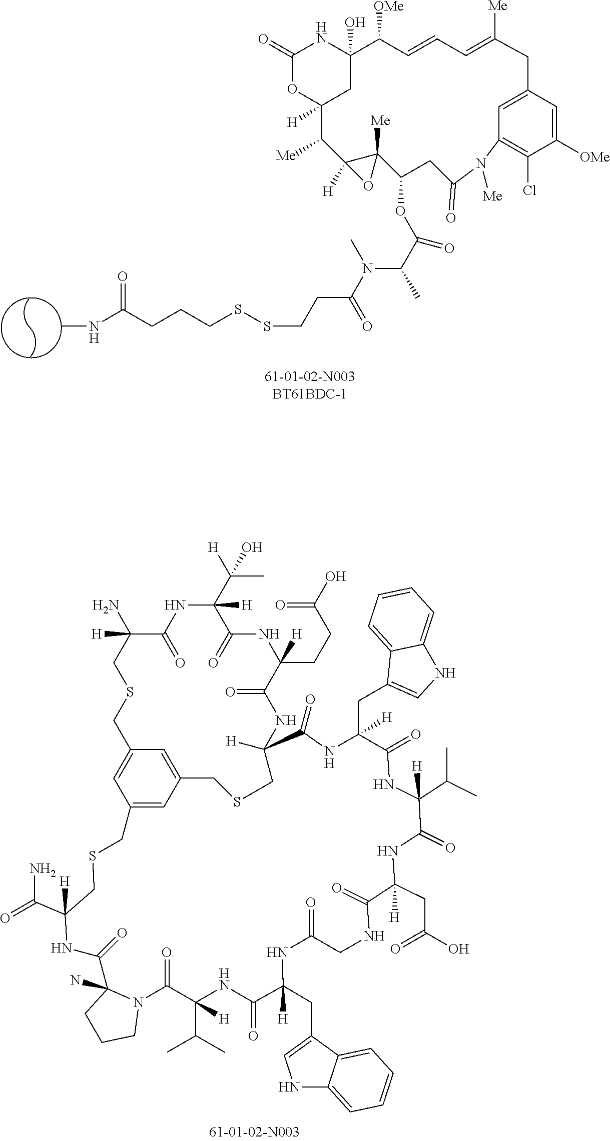 [SMILES: CNC(=O)CCCSSCCC(=O)N(C)[C@@H](C)C(=O)O[C@H]1CC(=O)N(C)c2cc(cc(OC)c2Cl)C/C(C)=C/C=C/[C@@H](OC)[C@@]2(O)C[C@H](OC(=O)N2)[C@@H](C)[C@@H]2O[C@@]12C ; CC(C)[C@H]1NC(=O)[C@H](Cc2c[nH]c3ccccc23)NC(=O)[C@@H]2CSCc3cc(cc(c3)CSC[C@H](N)C(=O)N[C@H]([C@@H](C)O)C(=O)N[C@H](CCC(=O)O)C(=O)N2)CSC[C@@H](C(N)=O)NC(=O)[C@@]2(N)CCCN2C(=O)[C@@H](C(C)C)NC(=O)[C@@H](Cc2c[nH]c3ccccc23)NC(=O)CNC(=O)[C@H](CC(=O)O)NC1=O]

Reaction Scheme:

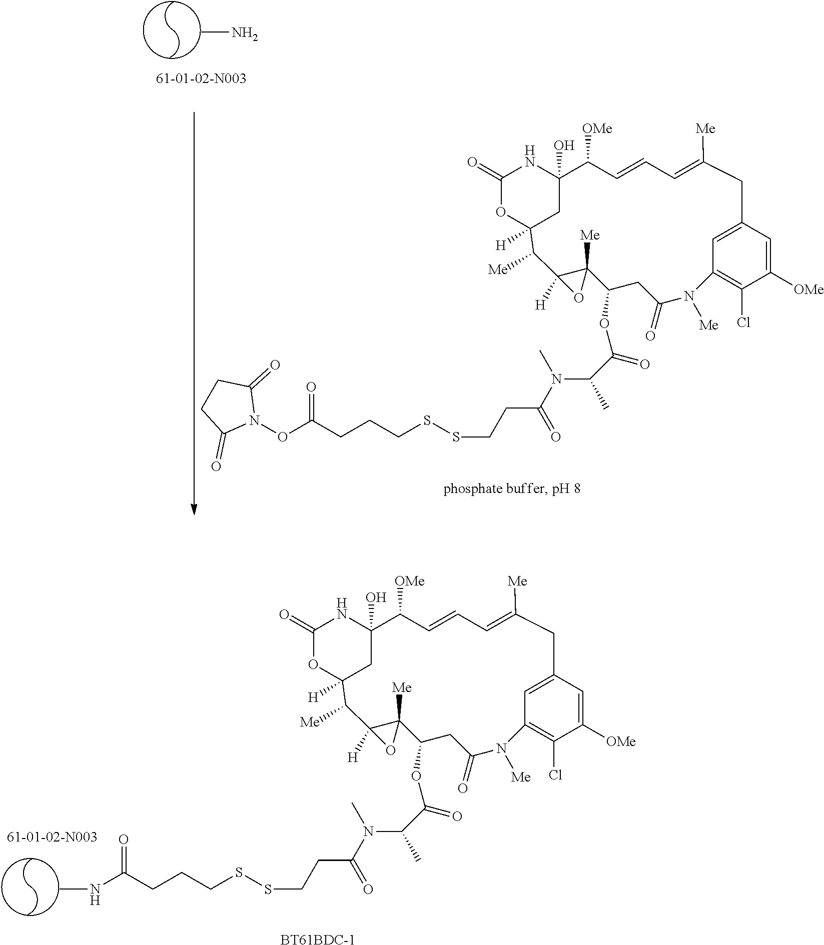

In a 50 mL conical tube, containing a stirbar, a 5 mg/mL solution of peptide 61-01-02-N003 (58 mg) was prepared using 11.6 mL sodium phosphate buffer (100 mM, pH 8.0). To this stirring solution was first added 500 μL DMSO, then after 5 minutes of additional stirring, the NHS-ester derivative of the payload (687 μL, 50 mM in DMSO) was added via pipetman. The resultant cloudy solution was stirred at room temperature. After 45 minutes the reaction continued to be cloudy, therefore 3.0 mL AcN was added to the reaction and the mixture became a clear solution. The reaction progress was monitored by LC/MS and was deemed complete after 3 hrs. The desired product was obtained via direct purification of the reaction mixture using a 50 g RediSep® Rf reversed phase C18 column and a solvent gradient of 5% B→95% B over 22 minutes. Pure fractions were combined and lyophilized to give 61.1 mg (78% yield) of the desired product as a fluffy white solid. Analysis by LC/MS indicated the material was at >95% purity (retention time: 2.95 min).

LC/MS (ES⁺) calc for [M-H₂O]⁺/2 1593; found 1593.

Prior to shipment, 61.1 mg of BT61BDC-1 was dissolved in 611 μL DMSO and split into 5 Eppendorf tubes (122.2 μL each, @ 100 mg/mL). One of these aliquots was then diluted to a concentration of 10 mg/ml via the addition of 1099.8 μL DMSO. The 10 mg/mL stock was then divided equally into 10 Eppendorf tubes (122.2 μL each, @ 10 mg/mL).

Biological Data

CAIX Competition Binding Assay

Affinity of the peptides of the invention for human CAIX (Ki) was determined using a competition fluorescence polarisation assay analogous to that described in Dubois et al (2011) Radiotherapy and Oncology 99(3), 424-43 using A-(SEQ ID NO:1)-A-Sar₆-K(FI) as the fluorescent ligand.

CAIX Enzyme Inhibition Assay

Enzyme inhibition was determined by a method analogous to that described in Hovanky et al (2014) Journal of Young Investigators 27 (2), 1-10.

The peptide ligands of the invention were tested in the above mentioned CAIX competition binding and enzyme inhibition assays and the results are shown in Table 1:

TABLE 1
Biological Assay Data for Peptide Ligands of the Invention

| | Molecular | | | Enzyme |
|---|---|---|---|---|
| Peptide | Scaffold | Ki (nM) | | IC₅₀ (nM) |
| 61-01-N001 | TBMB | 16.5 ± 2.47 | | |
| 61-01-N005 | TBMB | 35.2 | n = 1 | |
| 61-01-N003 | TBMB | 40 | n = 1 | |
| 61-01-01-N001 | TBMB | 81.45 ± 10.88 | | |
| 61-01-02-N001 | TBMB | 7.13 ± 2.29 | | 815 |
| 61-01-02-N002 | TBMB | 9.65 ± 3.63 | | |
| 61-01-02-N003 | TBMB | 9.99 ± 2.32 | | |
| 61-01-02-N004 | TBMB | 13.4 | n = 1 | |
| 61-01-02-N006 | TBMB | 7.3 ± 4.12 | | |
| 61-01-03-N001 | TBMB | 22.7 ± 0.98 | | |
| 61-01-04-N001 | TBMB | 5.77 ± 3.12 | | 855 |
| 61-01-04-N002 | TBMB | 13.1 | n = 1 | |
| 61-01-05-N001 | TBMB | 12.2 | n = 1 | |
| 61-01-06-N001 | TBMB | 15.3 | n = 1 | |
| 61-01-07-N001 | TBMB | 7.9 ± 0.98 | | |
| 61-01-08-N001 | TBMB | 5.6 | n = 1 | |
| 61-01-09-N001 | TBMB | 39.8 | n = 1 | |
| 61-18-N001 | TBMB | 312 | n = 1 | |

The bicycle drug conjugate of BT61BDC-1 was tested in the above mentioned CAIX competition binding assay and the results are shown in Table 2:

TABLE 2
Biological Assay Data for Bicycle Drug Conjugates of the Invention

| Bicycle Drug | | |
|---|---|---|
| Conjugates | Structure | Ki (nM) |
| BT61BDC-1 | 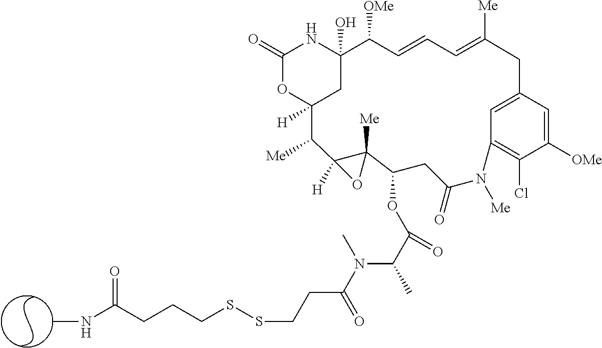 [SMILES: CNC(=O)CCCSSCCC(=O)N(C)[C@@H](C)C(=O)O[C@H]1CC(=O)N(C)c2cc(cc(OC)c2Cl)C/C(C)=C/C=C/[C@@H](OC)[C@@]2(O)C[C@H](OC(=O)N2)[C@@H](C)[C@@H]2O[C@@]12C] | 6.9* |
| | 61-01-02-N003 | |

*n = 1

1. A peptide ligand specific for CAIX comprising a polypeptide comprising at least three cysteine residues, separated by at least two loop sequences, and an aromatic molecular scaffold which forms covalent bonds with the cysteine residues of the polypeptide such that at least two polypeptide loops are formed on the molecular scaffold.

2. The peptide ligand as defined in claim 1, wherein said loop sequences comprise 2 or 7 amino acids.

3. The peptide ligand as defined in claim 1, wherein said loop sequences comprise three cysteine residues separated by two loop sequences one of which consists of 2 amino acids and the other of which consists of 7 amino acids.

4. The peptide ligand as defined in claim 1, wherein the polypeptide comprises an amino acid sequence selected from:
(SEQ ID NO: 12)
Cᵢ-X₁-X₂-Cᵢᵢ-W-I/A/V-D-G-W-V/I/M-P/S/E-Cᵢᵢᵢ;
and
(SEQ ID NO: 11)
Cᵢ-W-W-Cᵢᵢ-E-D-I-T-G-R-P-Cᵢᵢᵢ
wherein X₁ and X₂ represent any amino acid residue, and Cᵢ, Cᵢᵢ and Cᵢᵢᵢ represent first, second and third cysteine residues, respectively or a pharmaceutically acceptable salt thereof.

5. The peptide ligand as defined in claim 4, wherein the polypeptide comprises an amino acid sequence:
(SEQ ID NO: 12)
Cᵢ-X₁-X₂-Cᵢᵢ-W-I/A/V-D-G-W-V/I/M-P/S/E-Cᵢᵢᵢ
wherein X₁-X₂ represent any amino acid residue, X₃ is either absent or represents any amino acid, one of X₄ and X₅ represents any amino acid and the other is absent and Cᵢ, Cᵢᵢ and Cᵢᵢᵢ represent first, second and third cysteine residues, respectively or a pharmaceutically acceptable salt thereof.

6. The peptide ligand as defined in claim 4, wherein the amino acid sequence Cᵢ-X₁-X₂-Cᵢᵢ-W-I/A/V-D-G-W-V/I/M-P/S/E-Cᵢᵢᵢ (SEQ ID NO: 12) is selected from SEQ ID NOS: 1 to 10:
(SEQ ID NO: 1)
CᵢAECᵢᵢHWIDGWVPCᵢᵢᵢ;
(SEQ ID NO: 2)
CᵢGECᵢᵢWADGWVPCᵢᵢᵢ;
(SEQ ID NO: 3)
CᵢTECᵢᵢWVDGWVPCᵢᵢᵢ;
(SEQ ID NO: 4)
CᵢSECᵢᵢWVDGWVSCᵢᵢᵢ;
(SEQ ID NO: 5)
CᵢLECᵢᵢWVDGWVPCᵢᵢᵢ;
(SEQ ID NO: 6)
CᵢEECᵢᵢWVDGWVPCᵢᵢᵢ;
(SEQ ID NO: 7)
CᵢAECᵢᵢHWIDGWVECᵢᵢᵢ;
(SEQ ID NO: 8)
CᵢTECᵢᵢWIDGWVPCᵢᵢᵢ;
(SEQ ID NO: 9)
CᵢRECᵢᵢWVDGWVECᵢᵢᵢ;
and
(SEQ ID NO: 10)
CᵢGECᵢᵢWVDGWVSCᵢᵢᵢ;
wherein Cᵢ, Cᵢᵢ and Cᵢᵢᵢ represent first, second and third cysteine residues, respectively, or a pharmaceutically acceptable salt thereof.

7. The peptide ligand as defined in claim 6, wherein the amino acid sequence Cᵢ-X₁-X₂-Cᵢᵢ-W-I/A/V-D-G-W-V/I/M-P/S/E-Cᵢᵢᵢ (SEQ ID NO: 12) is selected from:
A-(SEQ ID NO: 1)-A (herein referred to as 61-01-N001);
G-Sar₁₀-A-(SEQ ID NO: 1) (herein referred to as 61-01-N005);
A-(SEQ ID NO: 1)-A-Sar₆-K-Biotin (herein referred to as 61-01-N003);
A-(SEQ ID NO: 2)-A (herein referred to as 61-01-01-N001);
A-(SEQ ID NO: 3)-A (herein referred to as 61-01-02-N001);
G-Sar₁₀-A-(SEQ ID NO: 3)-A (herein referred to as 61-01-02-N002);
β-Ala-Sarm-A-(SEQ ID NO: 3) (herein referred to as 61-01-02-N003);
Ac-A-(SEQ ID NO: 3)-A-Sar₁₀-D-K (herein referred to as 61-01-02-N004);
DOTA-β-Ala-Sar₁₀-A-(SEQ ID NO: 3) (herein referred to as 61-01-02-N006);
A-(SEQ ID NO: 4)-A (herein referred to as 61-01-03-N001);
A-(SEQ ID NO: 5)-A (herein referred to as 61-01-04-N001);
G-Sar₁₀-A-(SEQ ID NO: 5) (herein referred to as 61-01-04-N002);
A-(SEQ ID NO: 6)-A (herein referred to as 61-01-05-N001);
A-(SEQ ID NO: 7)-A (herein referred to as 61-01-06-N001);
A-(SEQ ID NO: 8)-A (herein referred to as 61-01-07-N001);
A-(SEQ ID NO: 9)-A (herein referred to as 61-01-08-N001); and
A-(SEQ ID NO: 10)-A (herein referred to as 61-01-09-N001).

8. The peptide ligand as defined in claim 1, wherein the molecular scaffold is 1,3,5-tris(bromomethyl)benzene (TBMB) and the polypeptide comprises an amino acid sequence selected from:
A-(SEQ ID NO: 1)-A (herein referred to as 61-01-N001);
G-Sar₁₀-A-(SEQ ID NO: 1) (herein referred to as 61-01-N005);
A-(SEQ ID NO: 1)-A-Sar₆-K-Biotin (herein referred to as 61-01-N003);
A-(SEQ ID NO: 3)-A (herein referred to as 61-01-02-N001);
G-Sar₁₀-A-(SEQ ID NO: 3)-A (herein referred to as 61-01-02-N002);
β-Ala-Sarm-A-(SEQ ID NO: 3) (herein referred to as 61-01-02-N003);
Ac-A-(SEQ ID NO: 3)-A-Sar₁₀-D-K (herein referred to as 61-01-02-N004);
DOTA-β-Ala-Sar₁₀-A-(SEQ ID NO: 3) (herein referred to as 61-01-02-N006);
A-(SEQ ID NO: 4)-A (herein referred to as 61-01-03-N001);
A-(SEQ ID NO: 5)-A (herein referred to as 61-01-04-N001);
G-Sar₁₀-A-(SEQ ID NO: 5) (herein referred to as 61-01-04-N002);
A-(SEQ ID NO: 6)-A (herein referred to as 61-01-05-N001);
A-(SEQ ID NO: 7)-A (herein referred to as 61-01-06-N001);
A-(SEQ ID NO: 8)-A (herein referred to as 61-01-07-N001);
A-(SEQ ID NO: 9)-A (herein referred to as 61-01-08-N001);
A-(SEQ ID NO: 10)-A (herein referred to as 61-01-09-N001); and
A-(SEQ ID NO: 11)-A (herein referred to as 61-18-N001).

9. The peptide ligand as defined in claim 1, wherein the molecular scaffold is 1,3,5-tris(bromomethyl)benzene (TBMB) and the polypeptide comprises an amino acid sequence:
β-Ala-Sar₁₀-A-(SEQ ID NO: 3) (herein referred to as 61-01-02-N003).

10. The peptide ligand as defined in claim 1, wherein the pharmaceutically acceptable salt is selected from the free acid or the sodium, potassium, calcium, or ammonium salt.

11. The peptide ligand as defined in claim 1, wherein the CAIX is human CAIX.

12. A drug conjugate comprising the peptide ligand as defined in claim 1, conjugated to one or more effector and/or functional groups.

13. The drug conjugate as defined in claim 12, wherein the one or more effector and/or functional groups are cytotoxic agents.

14. The drug conjugate as defined in claim 13, wherein said cytotoxic agent is DM-1.

15. The drug conjugate as defined in claim 13, wherein said cytotoxic agent is DM-1 and the peptide ligand is βAla-Sar₁₀-A-(SEQ ID NO: 3) (herein referred to as 61-01-02-N003)), and wherein the drug conjugate is of the formula:

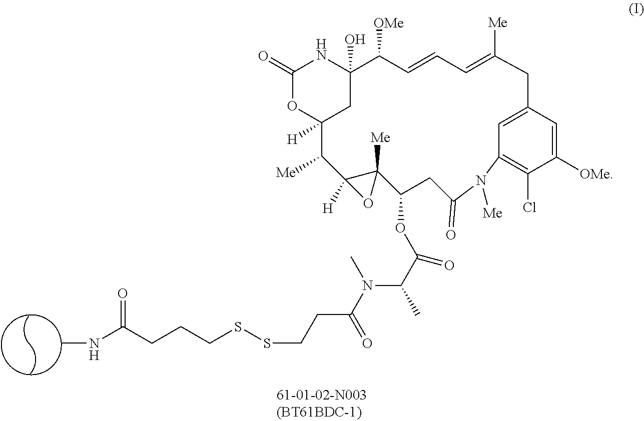 [SMILES: CNC(=O)CCCSSCCC(=O)N(C)[C@@H](C)C(=O)O[C@H]1CC(=O)N(C)c2cc(cc(OC)c2Cl)C/C(C)=C/C=C/[C@@H](OC)[C@@]2(O)C[C@H](OC(=O)N2)[C@@H](C)[C@@H]2O[C@@]12C]

16. A pharmaceutical composition which comprises the peptide ligand of claim 1, in combination with one or more pharmaceutically acceptable excipients.

17. A method for preventing, suppressing or treating a disease or disorder mediated by CAIX in a patient, comprising administering to the patient the peptide ligand as defined in claim 1.

18. A pharmaceutical composition which comprises the drug conjugate as defined in claim 12, in combination with one or more pharmaceutically acceptable excipients.

19. A method for preventing, suppressing or treating a disease or disorder mediated by CAIX in a patient, comprising administering to the patient the drug conjugate as defined in claim 12.
